(19) United States
(12) Patent Application Publication
(10) Pub. No.: US 2016/0174545 A1
(43) Pub. Date: Jun. 23, 2016

(54) Closed System Cryopreservation Device

(71) Applicant: Biotech Incorporated, Alpharetta, GA (US)

(72) Inventors: Jorge E. Parra, Cumming, GA (US); Diana P. Bernal, Cumming, GA (US)

(21) Appl. No.: 14/577,578

(22) Filed: Dec. 19, 2014

(51) Int. Cl. A01N 1/02

(52) U.S. Cl. CPC A01N 1/0257

(57) ABSTRACT

A closed system cryopreservation device for vitrification of biological specimens includes an elongated body from one end of which extends a frustoconical boss and an elongated cap for sealably enclosing a biological specimen with an elongated hollow chamber. The interior surface of the chamber defines a frustoconical volume corresponding to the frustoconical boss, such that when the boss is inserted into the chamber, substantially the entire interior surface is in contact with the exterior surface of the boss. The device further comprises a substantially uniform coefficient of thermal expansion.

### BACKGROUND

1. Field

The device described and claimed herein is in the field of devices for the cryopreservation of biological specimens.

2. Description of the Problem and Related Art

Cryopreservation is practiced in the life sciences for the purpose of halting biological activity in valuable cells for an extended period of time. Among the techniques used for cryopreservation is vitrification.

Vitrification involves the transformation of a solution comprised of a biological specimen, i.e., an oocyte or an embryo, into a glass-like amorphous solid that is free from any crystalline structure, followed by extremely rapid cooling. One of the major challenges of this method is to prevent the intracellular liquid within the oocyte or embryo to form ice crystals. Accordingly, the first step is to dehydrate the cell or cells as much as possible using cryoprotectant containing fluids called “vitrification media.” The biological specimen is then rapidly chilled by immersion in a cryogenic fluid such as liquid nitrogen (LN₂). With a proper combination of chilling speed and cryoprotectant concentration, intracellular water will attain a solid, innocuous, glassy (vitreous) state rather than an orderly, damaging, crystalline ice state. Vitrification can be described as a rapid increase in fluid viscosity that traps the water molecules in a random orientation. Vitrification media, however, can contain relatively high levels of cryoprotectant that can be toxic to cells except in the vitreous state. As a result, the time exposure of cells to vitrification media during dehydration and warming must be carefully controlled to avoid cellular injury, and, accordingly, it is desirable to chill the specimen as quickly as possible.

This impetus led to development of a method in which the biological specimen is directly immersed in the cryogen to achieve rapid chilling. Cryocontainer devices used in this technique are classified as “open” for use in an “open system” because the biological specimen is in direct contact with the cryogen, e.g., LN₂. Examples include electron microscopy grids, open pulled straws, the Cryoloop™, from Hampton Research Corp., of Aliso Viejo, Calif., USA, and Cryotop® offered by KitaZato Biopharma Co. Ltd, of Fuji, Shizuoka, Japan. Open carriers also enable rapid warming of the biological specimen.

LN₂, however, is not aseptic. It may contain bacterial and fungal species, which are viable upon warming. Furthermore, it has been reported that vitrified cells held in long term storage in LN₂ could be infected by viral pathogens artificially placed in said LN₂. Hence, there is the potential for infection of biological samples vitrified in open carriers. As a result, many countries have banned open systems due to the high risk of sample contamination.

The potential of infection has led to the development of sealed cryocontainers where the biological sample is placed in a cryocontainer and sealed before chilling in LN₂. The cryocontainer also serves as a storage device to isolate it from the cryogen during long-term storage. A “closed” system refers to a vitrification system that prevents direct contact between LN₂ and the biological material. Examples of closed cryocontainers include Cryotip®, offered by Irvine Scientific, and the “Cryotop® SC” from KitaZato. In both cases, the containers are heat-sealed to enclose the specimen.

Another example of a cryocontainer device for use in closed system, U.S. Pat. No. 7,316,896, to Kuwayama, et al., “Egg freezing and storing tool and method”, describes a closed cryocontainer for vitrification. This device comprises a fine plastic tube (nominally 0.25 mm OD and a wall thickness of 0.02 mm). A typical biological specimen will contain a human oocyte having an OD of 0.125 mm. It is dehydrated with vitrification media and then drawn into the tube. Then both ends of the tube are heat-sealed with a thermal sealing device to create an aseptic container. Because one of the heat seals is created very close to the biological specimen, there are concerns that the heat will injure the cell.

Similarly, U.S. Pat. No. 8,372,633, “Kit for Packaging Predetermined Volume of Substance to be Preserved by Cryogenic Vitrification”, to Clairaz, et al., describes a tube-within-a-tube closed cryocontainer concept. Both tubes are fabricated from plastic. The inner tube is modified to create a channel at one end upon which the biological specimen is placed. The loaded inner tube is then placed within the outer tube. The outer tube is then heat-sealed at the loading end to create an aseptic cryocontainer. However, heat-sealing requires a costly sealing device capable of fusing the plastic of a vitrification cryocontainer. It also adds another step in a process that requires speed for safe execution.

To address this short-coming, a closed system container device is presented by U.S. Pub. App. 20090123996, by Chin, and entitled, “Vitrification Device With Shape Memory Seal.” The device is disclosed to comprises a specimen collection tube in one end of which a stopper is installed. The collection tube, with specimen, is inserted into a tubular sheath until the stopper engages the sheath. Then, instead of heat-sealing, a separate closing device is installed on the stoppered sheath. The closing device comprises a cap that is drawn down on the stopper by a shape memory material that contracts when subjected to low temperatures, such as when immersed in LN₂. A problem with this approach is that it increases the complexity of the cryocontainer device, and because the components are made of different materials, each having its own coefficient of thermal expansion, they expand or contract at different rates which may also disrupt the seal.

### BRIEF DESCRIPTION OF THE DRAWINGS

The present device is described with reference to the accompanying drawings. In the drawings, like reference numbers indicate identical or functionally similar elements.

FIG. 1 is a side view of an exemplary embodiment of the disclosed cryopreservation device;

FIG. 2A illustrates a stick member of

FIG. 2B illustrates a cap for the embodiment shown in FIG. 1;

FIG. 3A is a detailed view of one end of the stick member;

FIG. 3B a detailed view of the end of the stick member shown in FIG. 3A, with the stick member rotated 90° about the long axis;

FIG. 4 is a detailed, fragmented view illustrating the cap engaged on the stick member; and

FIGS. 5A through 5D show various exemplary shapes that may comprise the perpendicular cross-section of the device.

### DETAILED DESCRIPTION

The various embodiments of the closed system cryopreservation storage device describe below and their advantages are best understood by referring to FIGS. 1 through 5D of the drawings. The elements of the drawings are not necessarily to scale, emphasis instead being placed upon clearly illustrating the novel features and principles of operation. Throughout the drawings, like numerals are used for like and corresponding parts of the various drawings.

Furthermore, reference in the specification to “an embodiment,” “one embodiment,” “various embodiments,” or any variant thereof means that a particular feature or aspect described in conjunction with the particular embodiment is included in at least one embodiment. Thus, the appearance of the phrases “in one embodiment,” “in another embodiment,” or variations thereof in various places throughout the specification are not necessarily all referring to its respective embodiment.

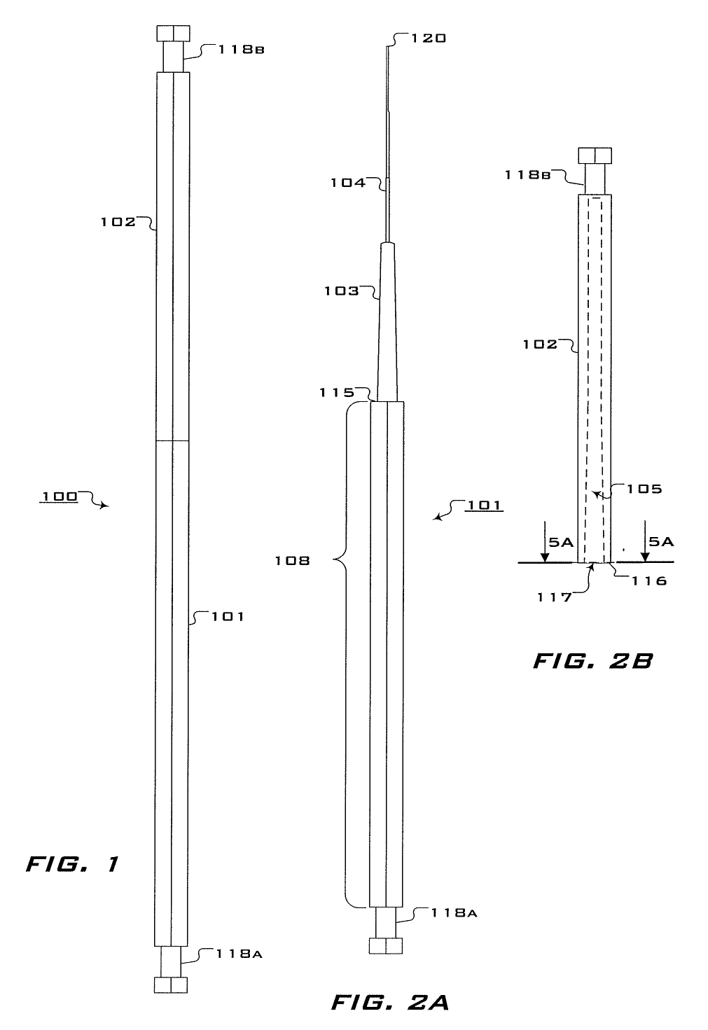

Referring to FIGS. 1, 2A & 2B, a closed system cryopreservation device 100 comprises an elongated stick 101 and a cap 102. The stick comprises a body 108 having a generally uniform cross-section abruptly transitioning at roughly midway along the stick 101 to a frustoconical boss 103. A shoulder 115 having a generally planar surface oriented roughly perpendicularly to the long axis of the stick 101 is formed at the transition from the body 108 to the boss 103. A specimen collection tip 104 extends from the narrow end of the boss 103. The cap 102 comprises an open end 116 having a generally planar surface in which is defined a circular opening 117. The opening 117 is in communication with an elongated hollow chamber 105 defined along the long axis of the cap 102 and dimensioned to accommodate the tip 104 and the boss 103. The cap 102 preferably comprises the same cross-sectional shape as the stick 101, e.g., hexagonal (FIG. 5A), triangular (FIG. 5B), square (FIG. 5C, circular (FIG. 5D), or the like, and roughly equal cross-sectional dimensions. Additionally, an optional, advantageous structural feature is a circumferential notch 118a, b disposed near the ends of the stick 101 and the cap 102, respectively, by which the device 100 may be clasped with forceps, making the device 100 easier to hold the device 100.

When a specimen (oocyte or embryo) is to be vitrified, it is collected and processed according to, for example, the protocol described above, and then deposited on the specimen collection tip 104. The tip 104 is then inserted into the elongated chamber 105 through the opening 117 and the cap 102 is pressed into place, until the planar surface of the open end 116 is seated against the planar surface of the shoulder 115. Accordingly, it will be appreciated that one planar surface should be substantially parallel with the opposing planar surface.

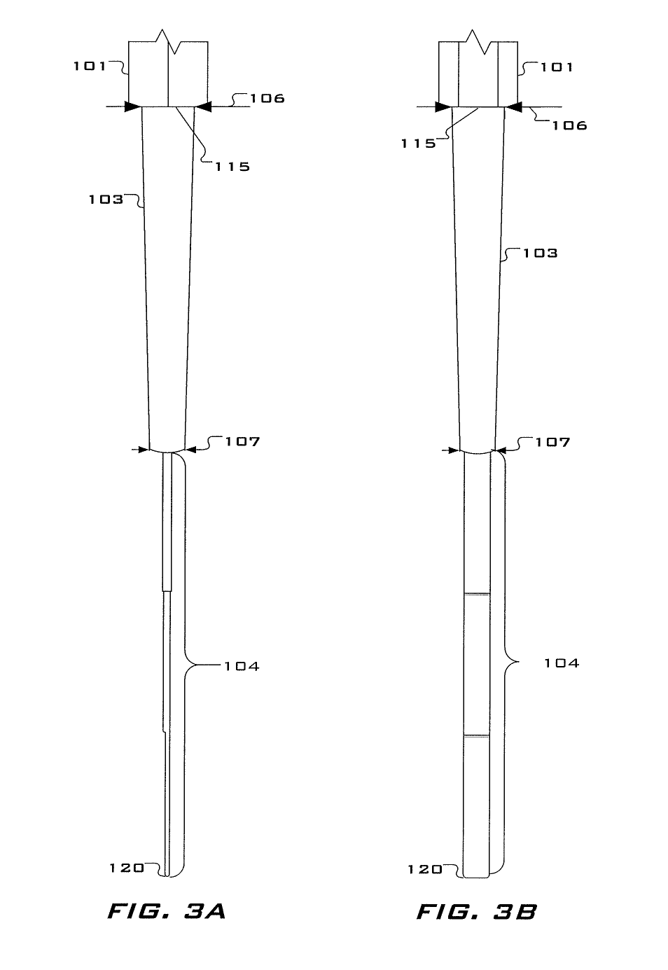

FIGS. 3A & 3B are detailed views, one rotated 90° from the other, of the portion of the stick 101 comprising the boss 103 and the tip 104. The boss 103 comprises a first outside diameter 106 (O.D.) at the base of the frustum adjacent the shoulder 115, and a second O.D. 107 at the distal end of the boss 103.

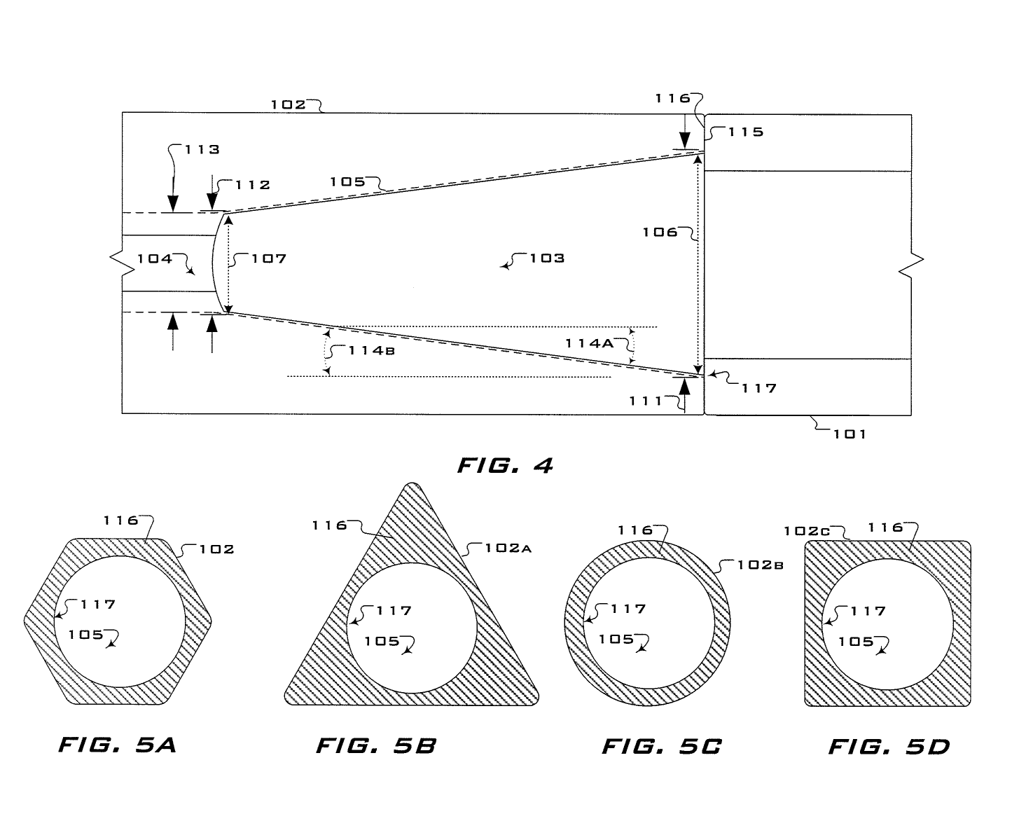

Referring now to FIG. 4, the cap 102 is shown seated against the shoulder 115 of the stick member 101 such that the tip 104 and the boss 103 are housed within the hollow chamber 105. It will be appreciated that the proportions illustrated in this view are exaggerated and not to scale to clearly show the dimensional features of the cap 102 and boss 103 and the inter-engagement of the two pieces. Accordingly, the proportions or dimensions that may be suggested in FIG. 4 are not to be construed as limiting any dimension to a particular value unless expressly defined herein.

As shown, boss 103 includes a first O.D. 106 that is greater than a second O.D. 107, the diameter of the boss tapering from the first O.D. 106 to the second O.D. 107 according to an angle 114A. The hollow chamber 105 comprises a first section defined from the open end 116 of the cap 102 and which is configured with a first inside diameter 111 (I.D.) located at the opening 117 and a second I.D. 112, such that the first I.D. 111 is greater than the second I.D. 112, decreasing according to angle 1114B. The second section of the chamber 105 comprises an elongated portion having a third I.D. 113 that is dimensioned to accommodate the tip 104. Therefore, the interior surface of the first section of the hollow chamber 105 defines a frustoconical space.

In this embodiment, angles 114A and 114B are roughly equal, preferably within a tolerance of 0.1%. The degree of taper should be relatively slight, no more than about 1.5°, and preferably about 0.50°. Thus, the first I.D. 111 is greater than the first O.D. 106, and the second I.D. 112 is greater than the second O.D. 107, in both cases by no more than about 0.1%. Accordingly, the frustoconical space defined by the interior surface of the hollow chamber 105 corresponds to the volume of the frustoconical boss 103 such that when the cap 102 is seated on the body 108, the specimen collection tip 104 is enclosed within the second section of the hollow chamber 105 and substantially all of the interior surface of the frustoconical section of the chamber 105 is in contact with the exterior surface of the boss 103. In this way, the hollow chamber 105 is sealed against entry of LN₂ when the cap 102 is properly installed without taking an additional step of heat-sealing the device.

Furthermore, there is also no need for a gasket. Typically such gaskets are comprised or a flexible, resilient material suitable for use with LN₂, such as silicon. However, silicon possesses a coefficient of thermal expansion different from the rigid material used to form the body and the cap.

On the other hand, the stick 101 and the cap 102 are made of the same rigid material which is suitable for immersion in cryogenic substances so that both pieces exhibit the same coefficient of thermal expansion. Various polymers may be used: polyester (for example, polyethylene terephthalate, polybutylene terephthalate); polyolefin (for example, polyethylene, ultra-high molecular-weight polyethylene, polypropylene, ethylene-propylene copolymer, ethylene-vinyl acetate copolymer), styrene resin (for example, polystyrene, methacrylate-styrene copolymer, methacrylate-butylene-styrene copolymer); and polyamide (for example, nylon 6, nylon 66). Preferably, both the stick 101 and the cap 102 are formed from a medical grade polystyrene crystal. Thus, the volumes of both pieces expand or contract in response temperature at the same rate insuring the interior surface of the frustoconical portion of the hollow chamber remains in substantially full contact with the exterior surface of the boss 103, maintaining the seal provided by the cap. Thus, the device maintains an equally secure seal both at room temperature and at low cryogenic temperatures, facilitating substantially uniform temperature conduction throughout the entire volume of the device.

As described above and shown in the associated drawings, the present invention comprises a closed system cryopreservation device. While particular embodiments have been described, it will be understood, however, that any invention appertaining to the device described is not limited thereto, since modifications may be made by those skilled in the art, particularly in light of the foregoing teachings. It is, therefore, contemplated by the appended claims to cover any such modifications that incorporate those features or those improvements that embody the spirit and scope of the invention.

What is claimed is:

1. A closed system cryopreservation device for vitrification of biological specimens, said cryopreservation device comprising:
an elongated body;
a frustoconical boss having an exterior surface and extending from a first end of said elongated body;
a specimen collection tip extending from said boss; and
an elongated cap for sealably enclosing said specimen collection tip, said cap having a first end in which an opening is defined that is in communication with an elongated hollow chamber extending along a long axis of said cap and having a length sufficient to accommodate said tip and said boss, said hollow chamber comprising an interior surface that defines a frustoconical volume corresponding to said frustoconical boss, such that when said boss is inserted into said hollow chamber, substantially all of said interior surface is in contact with said exterior surface of said boss; and
wherein said device comprises a substantially uniform coefficient of thermal expansion.

2. The closed system cryopreservation device of claim 1, further comprising at least one circumferential notch defined in either of said body or said cap.

3. The closed system cryopreservation device of claim 1, wherein said body comprises a long axis and a perpendicular cross-section shape, said shape being one of circular, rectangular, triangular, and hexagonal.

4. The closed system cryopreservation device of claim 1, wherein said body comprises a first long axis and a perpendicular cross-section shape and further comprising a shoulder disposed between the base of said frustroconical boss and having a first planar surface generally perpendicular to said first long axis.

5. The closed system cryopreservation device of claim 4, wherein said cap comprises a second long axis and a perpendicular cross-section shape, wherein said first end of said cap comprises a second planar surface generally perpendicular to said second long axis such that substantially all of said first planar surface of said shoulder is in contact with said second planar surface.

6. The closed system cryopreservation device of claim 1, wherein said body and said cap comprise the same material.

7. The closed system cryopreservation device of claim 6, wherein said body comprises a first long axis and a perpendicular cross-section shape and further comprising a shoulder disposed between the base of said frustoconical boss and having a first planar surface generally perpendicular to said first long axis.

8. The closed system cryopreservation device of claim 7, wherein said cap comprises a second long axis and a perpendicular cross-section shape, wherein said first end of said cap comprises a second planar surface generally perpendicular to said second long axis such that substantially all of said first planar surface of said shoulder is in contact with said second planar surface.

9. The closed system cryopreservation device of claim 8, further comprising a crystal polystyrene.

10. The closed system cryopreservation device of claim 1, further comprising a first angle defined by a decrease in diameter of said frustoconical boss and a second angle defined by a decrease in diameter of said frustoconical space, and wherein said first and second angles are substantially equal.

11. The closed system cryopreservation device of claim 10, wherein said first and second angles are no greater than about 1.5°.

12. The closed system cryopreservation device of claim 11, wherein said first and second angles are about 0.5°.

13. The closed system cryopreservation device of claim 12, wherein said body comprises a first long axis and a perpendicular cross-section shape and further comprising a shoulder disposed between the base of said frustroconical boss and having a first planar surface generally perpendicular to said first long axis.

14. The closed system cryopreservation device of claim 13, wherein said cap comprises a second long axis and a perpendicular cross-section shape, wherein said first end of said cap comprises a second planar surface generally perpendicular to said second long axis such that substantially all of said first planar surface of said shoulder is in contact with said second planar surface.

15. The closed system cryopreservation device of claim 1, wherein said frustoconical boss comprises an outside diameter and said frustoconical space comprises an inside diameter, and wherein said insider diameter is greater than said outside diameter by no more than about 0.1%.
